(19) United States
(12) Patent Application Publication
(10) Pub. No.: US 2026/0151090 A1
(43) Pub. Date: Jun. 4, 2026

(54) Catheter Sensor Array

(71) Applicants: TE Connectivity Solutions GmbH, Schaffhausen (CH); TE Sensores, S. DE R.L. DE C.V., Hermosilo (MX)

(72) Inventors: Michael Klitzke, Fremont, CA (US); Britta Aldejohann, Fremont, CA (US); Predrag Drljaca, Schaffhausen (CH); Ilie Poenaru, Bevaix (CH); Enrique Cedeno, Tlalnepantla (MX)

(73) Assignees: TE Connectivity Solutions GmbH, Schaffhausen (CH); TE Sensores, S. DE R.L. DE C.V., Hermosillo (MX)

(21) Appl. No.: 18/966,290

(22) Filed: Dec. 3, 2024

(51) Int. Cl. A61B 5/00; A61B 90/00

(52) U.S. Cl. CPC A61B 5/6859; A61B 90/06; A61B 2090/064; A61B 2560/0462; A61B 2562/0247; A61B 2562/0271; A61B 2562/066; A61B 2562/164; A61B 2562/227

(57) ABSTRACT

A catheter sensor array includes a catheter, a plurality of sensor elements positioned at a distal end of the catheter, and a plurality of sensor controllers each connected to at least one of the sensor elements by a first connection. Each of the sensor controllers is positioned along a length of the catheter closer to the distal end of the catheter than to a proximal end of the catheter opposite the distal end.

### FIELD OF THE INVENTION

The present invention relates to an array of sensors and, more particularly, to an array of sensors in a catheter.

### BACKGROUND OF THE INVENTION

Catheters with sensor arrays are commonly used in minimally invasive medical applications, such as for ablation, cardiac mapping, or manometry. The sensors are used to measure various physical quantities for the application, for example force, pressure, and temperature, and often incorporate a large number of sensors to measure a range of physical quantities in different locations. The sensors are positioned at the distal end of the catheter.

As the number of sensors in the sensor array of the catheter increases, a large number of leads are required to extend along the length of the catheter from the sensors to a controller at the proximal end, taking up space in the catheter that has strict dimensional requirements. The number and volume of leads limits the number of sensors that can be used in the catheter and limits the addition of other features and components in the catheter. Further, signals sent by the sensors along the leads extending from the distal end to the proximal end are subject to high levels of noise, which impairs the quality of data gathered by the catheter sensor array.

### SUMMARY OF THE INVENTION

A catheter sensor array includes a catheter, a plurality of sensor elements positioned at a distal end of the catheter, and a plurality of sensor controllers each connected to at least one of the sensor elements by a first connection. Each of the sensor controllers is positioned along a length of the catheter closer to the distal end of the catheter than to a proximal end of the catheter opposite the distal end.

### BRIEF DESCRIPTION OF THE DRAWINGS

The invention will now be described by way of example with reference to the accompanying Figures, of which:

FIG. 1 is a perspective view of a distal end of a catheter sensor array according to an embodiment;

FIG. 2 is a perspective view of the distal end of the catheter sensor array with a catheter transparent to show internal components of the catheter sensor array;

FIG. 3 is a schematic diagram of the catheter sensor array according to an embodiment;

FIG. 4 is a block diagram of a sensor controller and a subset of sensor elements;

FIG. 5 is a block diagram of a plurality of sensor elements and a plurality of sensor controllers connected to a main controller according to an embodiment;

FIG. 6 is a block diagram of a plurality of sensor elements and a plurality of sensor controllers connected to a main controller according to another embodiment;

FIG. 7 is a sectional side view of a sensor element connected to a flexible substrate according to an embodiment; and

FIG. 8 is a sectional side view of a sensor element connected to a flexible substrate according to another embodiment.

### DETAILED DESCRIPTION

Exemplary embodiments of the present disclosure will be described hereinafter in detail with reference to the attached drawings, wherein like reference numerals refer to like elements. The present disclosure may, however, be embodied in many different forms and should not be construed as being limited to the embodiments set forth herein; rather, these embodiments are provided so that the present disclosure will convey the concept of the disclosure to those skilled in the art. In addition, in the following detailed description, for purposes of explanation, numerous specific details are set forth to provide a thorough understanding of the disclosed embodiments. However, it is apparent that one or more embodiments may also be implemented without these specific details.

Throughout the drawings, only one of a plurality of identical elements may be labeled in a figure for clarity of the drawings, but the detailed description of the element herein applies equally to each of the identically appearing elements in the figure.

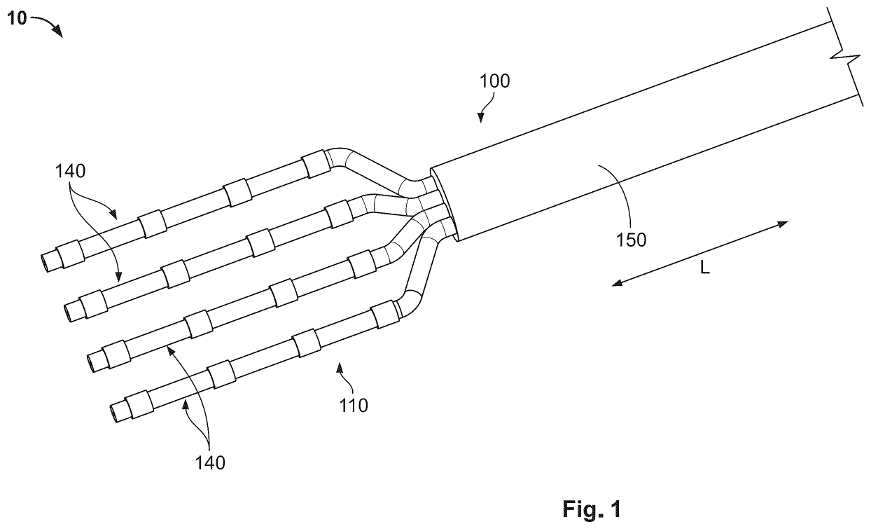

A catheter sensor array 10 according to various embodiments is shown in FIGS. 1-3. The catheter sensor array 10 includes a catheter 100, a plurality of sensor elements 200 disposed in the catheter 100, a plurality of sensor controllers 300 disposed in the catheter 100 and connected to the plurality of sensor elements 200, and a main controller 400 disposed in the catheter 100 and connected to the sensor controllers 300.

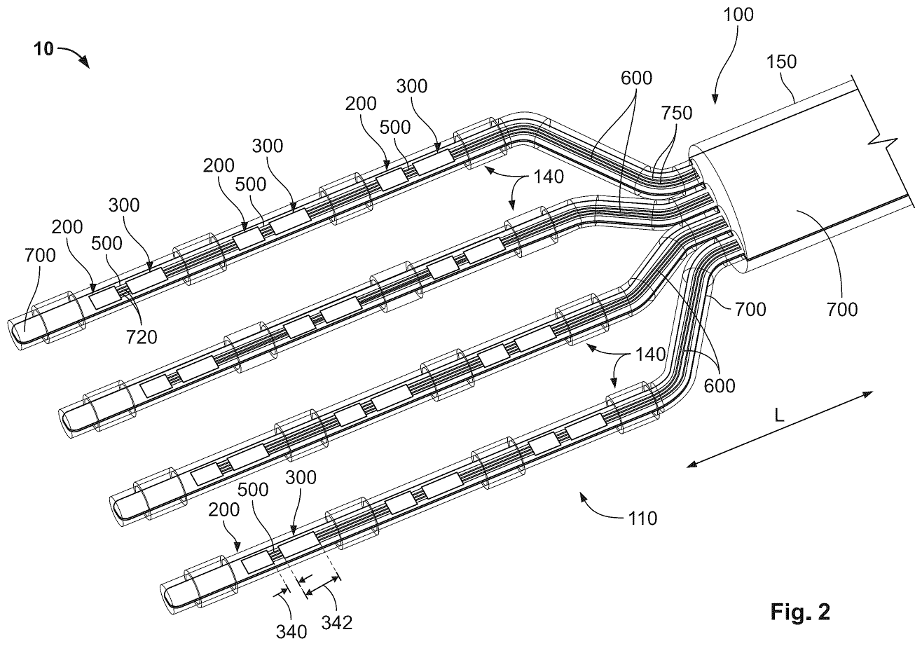

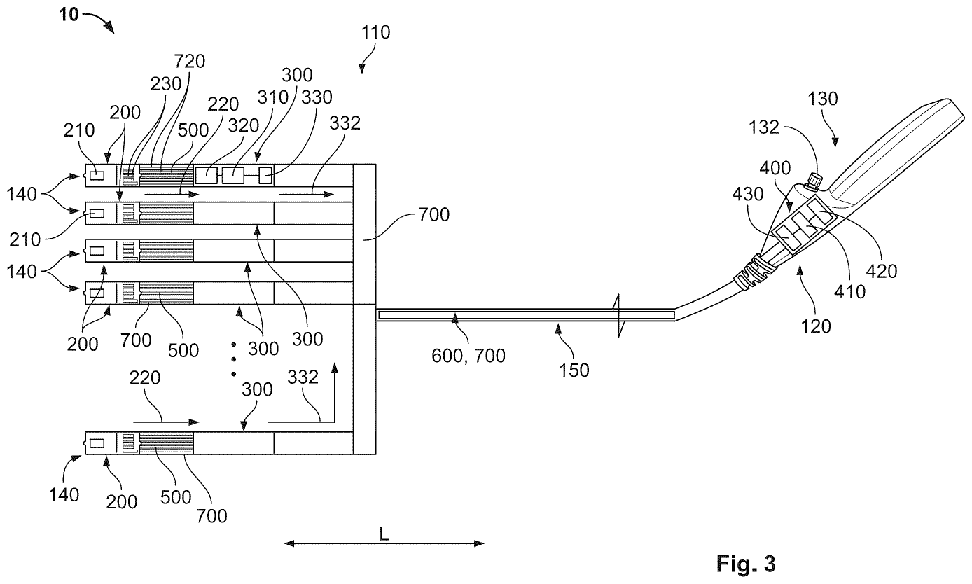

The catheter 100, as shown in FIGS. 1-3, has a distal end 110 and a proximal end 120 opposite the distal end 110 along a length L of the catheter 100. The distal end 110 is shown in detail in FIGS. 1 and 2. The catheter 100 has a handle 130 at the proximal end 120, shown in FIG. 3, a plurality of arms 140 at the distal end 110, and a lumen 150 extending between the handle 130 and the arms 140. In the shown embodiment, the lumen 150 leads to four arms 140 at the distal end 110. In other embodiments, the distal end 110 can have less than four arms 140, including one arm 140, or more than four arms 140.

The lumen 150 and the arms 140 are formed of a flexible material; the lumen 150 and the arms 140 may be formed of any material commonly used in catheter applications, such as for cardiac catheters. The lumen 150 and the arms 140 define interior spaces that extend along the length L of the catheter 100 and communicate with the handle 130.

An operator uses the handle 130 at the proximal end 120 to control a position of the catheter 100, including controlling a bending and positioning of each of the arms 140 in the present embodiment, using a control element 132 on the handle 130, as shown in FIG. 3. In the shown embodiment, the control element 132 is a knob. In other embodiments, the control element 132 may be any type of element used on the handle 130 of a catheter 100 to control the position of the distal end 110.

The sensor elements 200 may be any type of sensor used in catheter applications. In an embodiment, the sensor elements 200 each measure a pressure, a force, or a temperature. In the embodiments shown in FIGS. 3, 7, and 8, for example, the sensor elements 200 are each a full bridge micro-electromechanical system (MEMS) having a diaphragm 210 that deflects in proportion to a force or pressure applied on the diaphragm 210; the sensor element 200 outputs a sensor signal 220 representative of the force or pressure on the diaphragm 210. In other embodiments, the sensor elements 200 may measure any other type of physical quantity that is necessary or useful to measure in a catheter application and outputs the physical quantity as the sensor signal 220.

The sensor controllers 300, as shown in FIG. 3, each include a sensor processor 310, a sensor memory 320 connected to the sensor processor 310, and a sensor communication device 330 connected to the sensor processor 310. The sensor memory 320 is a non-transitory computer-readable medium that has a plurality of algorithms or computer programs stored thereon that, when executed by the sensor processor 310, perform the functions of the sensor controller 300 described herein. The sensor communication device 330 may be a wired or wireless communication device, as described in greater detail below. In an embodiment, the sensor controllers 300 are each an application-specific integrated circuit (ASIC); in this embodiment, the sensor processor 310 and the sensor memory 320 may be embodied as the ASIC on a single chip. In an embodiment, the ASIC that forms the sensor controller 300 may be an Application Specific Standard Part (ASSP).

FIGS. 2 and 3 schematically show the sensor elements 200 and sensor controllers 300 on each of the arms 140 of the catheter 100. In various embodiments, other passive and active electronic components may be present on each of the arms 140 to operate the sensor elements 200 and the sensor controllers 300, such as decoupling capacitors, inductors, resistors, amplifiers, or any other electronic elements that could be necessary for or aid in the function of the sensor elements 200 and the sensor controllers 300.

The main controller 400, shown in FIG. 3, includes a main processor 410, a main memory 420 connected to the main processor 410, and a main communication device 430 connected to the main processor 410. The main memory 420 is a non-transitory computer-readable medium that has a plurality of algorithms or computer programs stored thereon that, when executed by the main processor 410, perform the functions of the main controller 400 described herein. The main communication device 430 may be a wired or wireless communication device and, as described in greater detail below, communicates with the sensor communication devices 330 of the sensor controllers 300. In various embodiments, the main controller 400 including the main processor 410 and the main memory 420 can be separate components or integrated on a single chip.

As shown in FIGS. 2 and 3, the sensor elements 200 are positioned at the distal end 110 of the catheter 100. In the embodiment shown in FIG. 2, multiple sensor elements 200 are positioned in each of the arms 140 of the catheter 100, separated from one another along the length L of the catheter 100. In another embodiment depicted schematically in FIG. 3, one sensor element 200 is positioned at an end of each of the arms 140 along the length L. In other embodiments, the sensor elements 200 may be arranged in the arms 140 differently than in the embodiments shown, including with the sensor elements 200 unevenly distributed among the arms 140 or positioned differently in the arms 140 along the length L, provided that the sensor elements 200 in all embodiments are arranged at the distal end 110 of the catheter 100.

The sensor controllers 300, as shown in FIGS. 2 and 3, are each connected to at least one of the sensor elements 200 by a first connection 500. In the embodiment shown in FIGS. 2 and 3, each of the sensor controllers 300 is connected to one of the sensor elements 200 by the first connection 500 and outputs the sensor signal 220 representative of the measured physical quantity to the sensor controller 300 along the first connection 500.

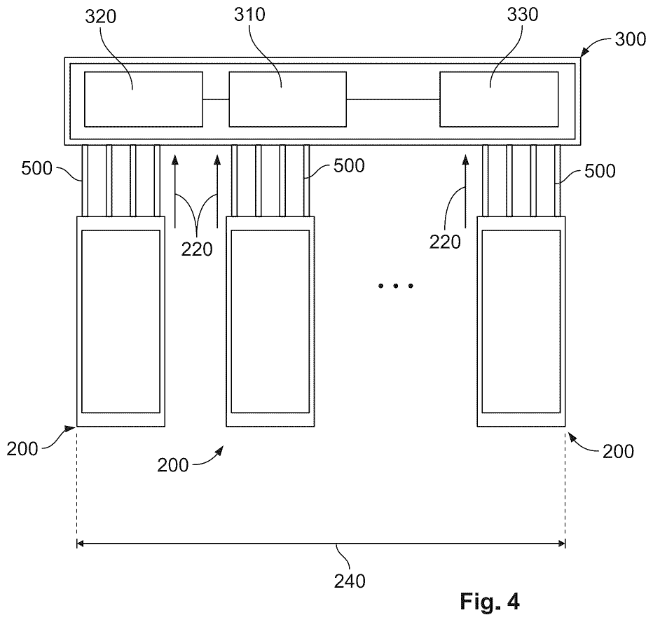

In another embodiment shown in FIG. 4, one of the sensor controllers 300 is connected to a subset 240 of the sensor elements 200. The subset 240 includes more than one of the sensor elements 200 each connected to the sensor controller 300 by the first connection 500; in the shown embodiment, the subset 240 includes three sensor elements 200 and, in other embodiments, could include two or more than three sensor elements 200. The sensor elements 200 in the subset 240 could all measure the same physical quantity, such as pressure, force, or temperature, or could measure different physical quantities. The sensor elements 200 in the subset 240 in FIG. 4 each output the sensor signal 220 representative of the measured physical quantity to the sensor controller 300. In various embodiments, the sensor controllers 300 may each be connected to a subset 240 of the sensor elements 200, may each be connected to only one of the sensor elements 200, or any combination thereof.

As shown in FIGS. 2 and 3, each of the sensor controllers 300 is positioned along the length L of the catheter 100 closer to the distal end 110 than to the proximal end 120. In the shown embodiment, each of the sensor controllers 300 is positioned at the distal end 110 of the catheter 100, in the arms 140 of the catheter 100. In an embodiment, each of the sensor controllers 300 is positioned within a close proximity of the sensor elements 200 to which the sensor controller 300 is connected. In the embodiment shown in FIG. 2, for example, the proximity 340 of the sensor controller 300 to the sensor element 200 connected by the first connection 500 along the length L of the catheter 100 is less than a largest dimension 342 of the sensor controller 300. In the embodiment shown in FIG. 4 in which the subset 240 of sensor elements 200 is connected to one sensor controller 300, the sensor controller 300 may be within close proximity 340 of each of the sensor elements 200 in the subset 240. In other embodiments, the proximity 340 of the sensor controller 300 and the connected sensor elements 200 may be greater than the largest dimension 342 of the sensor controller 300, provided that the sensor controllers 300 are positioned at the distal end 110 of the catheter 100 or at least closer to the distal end 110 than the proximal end 120.

The main controller 400 is positioned at the proximal end 120 of the catheter 100, as shown in FIG. 3. The main controller 400 is positioned in the handle 130 of the catheter 100 in the embodiment shown in FIG. 3. The main controller 400 is connected to each of the sensor controllers 300 by a second connection 600, shown in FIGS. 2 and 3, that extends from the main controller 400, through the lumen 150, and to the sensor controllers 300 at the distal end 100 of the catheter 100. As described in greater detail below, the sensor controllers 300 each output a digital signal 332 to the main controller 400 along the second connection 600.

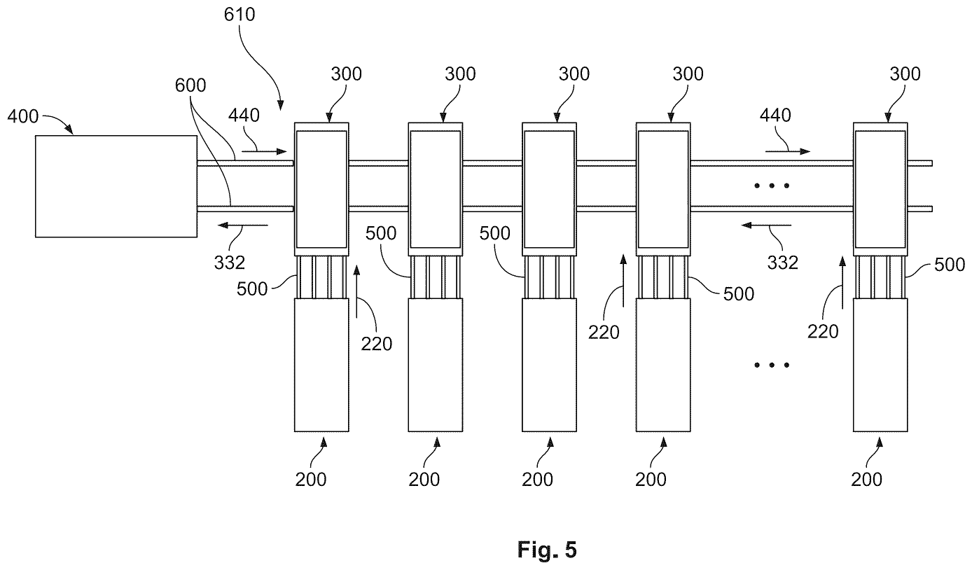

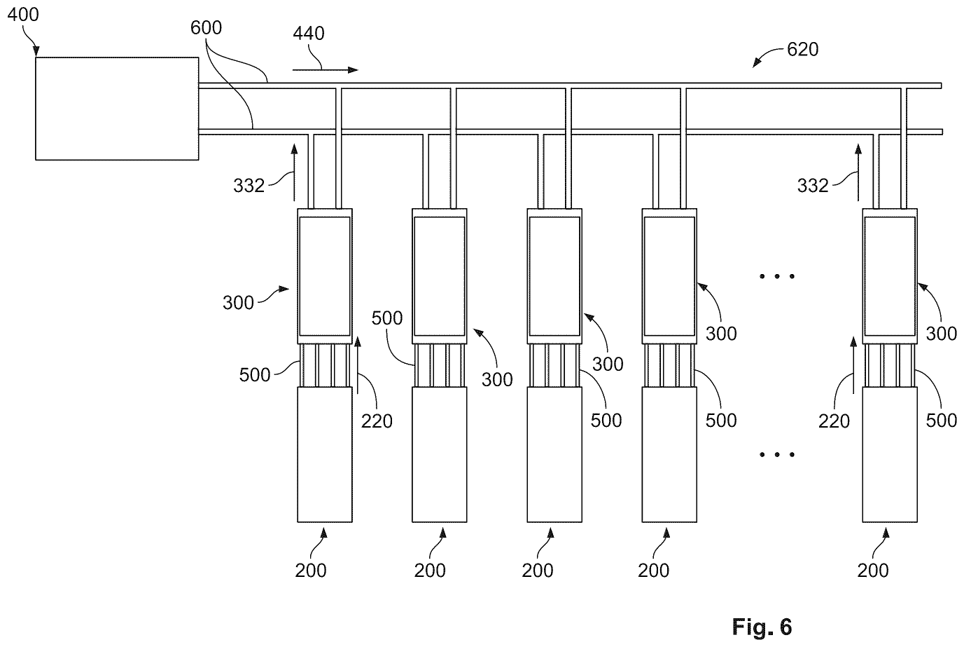

FIGS. 5 and 6 are schematic diagrams of various embodiments of the second connection 600 between the main controller 400 and the sensor controllers 300. In the embodiment shown in FIG. 5, the sensor controllers 300 are connected to the main controller 400 in a daisy chain 610 arrangement. In this embodiment, the sensor controllers 300 are each addressable but are connected through one another by the second connection 600 to transmit the digital signals 332 output by the sensor controllers 300 to the main controller 400 and receive controller signals 440 from the main controller 400. In the embodiment shown in FIG. 6, the sensor controllers 300 are connected to the main controller 400 in a digital bus 620 arrangement of the second connection 600. In this embodiment, each of the sensor controllers 300 is addressable and is separately connected to the main controller 400 by the second connection 600 to transmit the digital signals 332 output by the sensor controllers 300 to the main controller 400 and receive controller signals 440 from the main controller 400. In an embodiment, the main controller 400 can broadcast or simultaneously send controller signals 440 to all of the sensor controllers 300, prompting the sensor controllers 300 to each transmit the digital signals 332 to the main controller 400.

In the embodiment shown in FIGS. 2 and 3, the sensor elements 200 and the sensor controllers 300 are disposed on a flexible substrate 700. The flexible substrate 700 may be any type of flexible material that is used as a substrate for electrical connections and can be formed or adapted to have various shapes to fit various applications of the catheter 100. The flexible substrate 700 is positioned in the catheter 100 and extends, as shown in FIGS. 2 and 3, from the proximal end 120 along the lumen 150 and in each of the arms 140 of the catheter 100 at the distal end 110.

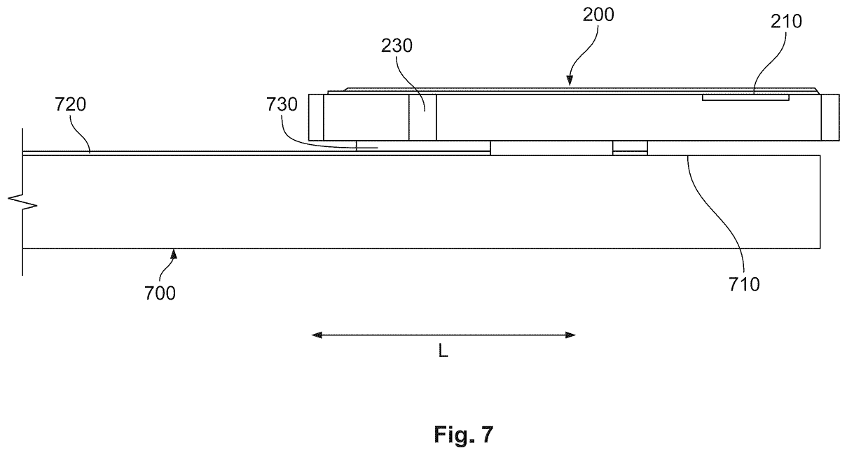

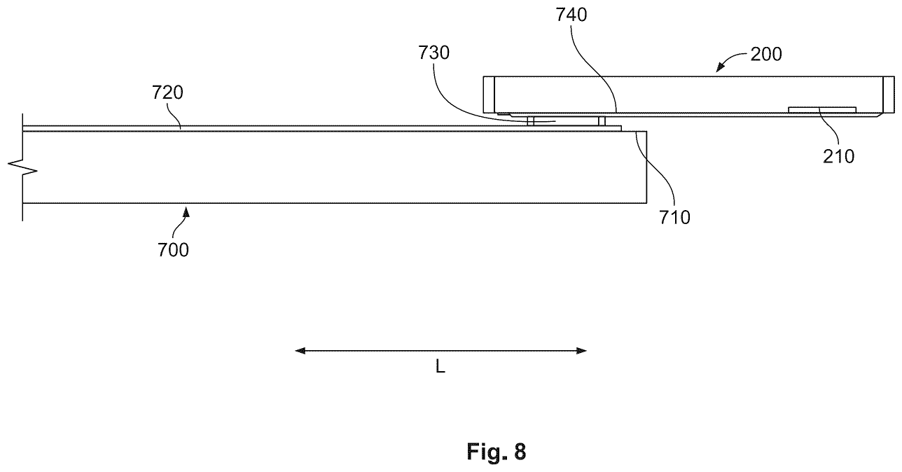

An attachment of the sensor element 200 to the flexible substrate 700 according to different embodiments is shown in FIGS. 7 and 8. The flexible substrate 700, in both embodiments, has a surface 710, a plurality of traces 720 disposed on the surface 710, and a solder bump 730 disposed on each of the traces 720. The traces 720 may be any type of electrically conductive material used on flexible substrates and the solder bumps 730 may be any type of solder material used to form electrical connections on flexible substrates. In other embodiments, other elements could be used in place of the solder bump 730, such as a gold ball, a molded stud, an electrically conductive polymer, a plated bump, or any other type of material used to mechanically and electrically connect a sensor element to a flexible substrate.

In the embodiment shown in FIG. 7, the sensor element 200 has the diaphragm 210 described above and a plurality of through silicon vias (TSV) 230 extending through the sensor element 200. The sensor element 200 is positioned on the surface 710 of the flexible substrate 700, with each TSV 230 positioned over one of the solder bumps 730 on one of the traces 720. The TSV 230 is filled with a conductive material and the solder bump 730 is reflowed to electrically connect the sensor element 200 to the trace 720 and mechanically connect the sensor element 200 to the flexible substrate 700. One TSV 230, one solder bump 730, and one trace 720 are shown in the sectional view of FIG. 7, but the sensor element 200 has a plurality of TSVs 230 connected to a plurality of traces 720, as shown in FIG. 3.

In another embodiment shown in FIG. 8, the sensor element 200 has a plurality of contact pads 740 instead of the TSVs 230. In this embodiment, the sensor element 200 is positioned on the surface 710 of the flexible substrate 700, with each of the contacts pads 740 positioned over one of the solder bumps 730 on one of the traces 720. The solder bump 730 is reflowed to electrically connect the sensor element 200 to the trace 720 and mechanically connect the sensor element 200 to the flexible substrate 700.

In other embodiments, the sensor element 200 can be electrically connected and mechanically attached to the flexible substrate 700 by any other possible form of electrical connection between a sensor and a flexible substrate, such as a wire bond connection, a beam lead connection, via a conductive epoxy, by tape automated bonding (TAB), or by bumped tape automated bonding (BTAB). The sensor controllers 300, in various embodiments, can be electrically connected and mechanically attached to the surface 710 of the flexible substrate 700 and the traces 720 by any of the same mechanisms or components disclosed for connecting the sensor elements 200.

In the embodiment shown in FIGS. 2, 3, 7, and 8, in which the sensor elements 200 and the sensor controllers 300 are positioned on the flexible substrate 700, the first connections 500 between the sensor elements 200 and the sensor controllers 300 are the traces 720 of the flexible substrate 700. In other embodiments, the flexible substrate 700 can be omitted, and the first connections 500 can be any other type of wired electrical connection between the sensor elements 200 and the sensor controllers 300 that is positioned in the arms 140 at the distal end 110 of the catheter 100. In these embodiments, three to five traces 720 or other wired electrical connection elements are required to connect each sensor element 200 to one of the sensor controllers 300. In other embodiments, less than three or more than five traces 720 may be used to connect each sensor element 200 to one of the sensor controllers 300.

In the embodiment shown in FIG. 2, the second connections 600 are a plurality of leads 750 on the flexible substrate 700 that extend between the sensor controllers 300 and the main controller 400. In this embodiment, the sensor controllers 300 require two to four leads 750 per sensor element 200 connected to the sensor controller 300 to transmit the physical quantity measured by the sensor element 200 to the main controller 400. In other embodiments, the sensor controllers 300 could require more than four leads 750 to connect to the main controller 400. In further embodiments, with or without the flexible substrate 700, the second connections 600 can be any type of wired connection, such as a multi-filar or fiber optic connection, or can be a wireless connection, such as a wi-fi or short-range radio connection.

During use of the catheter sensor array 10, the arms 140 at the distal end 110 of the catheter 100 are inserted into a body lumen or cavity, such as blood vessels or the heart, to measure and diagnose health concerns. In an exemplary embodiment, the catheter sensor array 10 shown in FIGS. 1 and 2 is a cardiac catheter used to evaluate the condition of a patient's heart by measuring forces, pressures, temperatures, and other physical quantities measurable by the sensor elements 200. The user manipulates the handle 132 at the proximal end 120 of the catheter 100 via the control element 132 to maneuver the arms 140 at the distal end 110. The arms 140 are maneuvered such that the sensor elements 200 are positioned at the sources of the physical quantities to be measured.

Each of the sensor elements 200 measures the desired physical quantity, for example by deflection of the diaphragm 210 as described above, and outputs a signal representative of the measured physical quantity as the sensor signal 220. The sensor signal 220 is output along the first connections 500 to the sensor controller 300 connected to the sensor element 200, as shown in FIGS. 3-6.

The sensor controller 300 receives the sensor signal 220 from the sensor element(s) 200 connected to the sensor controller 300. The sensor processor 310 converts the sensor signal(s) 220 into a digital signal 332 that is output by the sensor communication device 300. The sensor communication device 300 communicates with the main communication device 430, whether by a wired or wireless second connection 600 as described above, to output the digital signal 332 representing the physical quantities sensed by the sensor elements 200 to the main controller 400.

The sensor controllers 300, by gathering the sensor signals 220 from the sensor elements 200 and transmitting the digital signals 332 to the main controller 400, requires a number of second connections 600 in the catheter sensor array 10 that is less than a number of first connections 500 in the catheter sensor array 10. For example, in an embodiment in which the first connections 500 are traces 720 and the second connections 600 are leads 750, the number of traces 720 required to connect all the sensor elements 200 to the sensor controllers 300 is less than the number of leads 750 required to connect the sensor controllers 300 to the main controller 400. In another embodiment in which the sensor controllers 300 communicate wirelessly with the main controller 400, fewer connections are required along the length L of the catheter 100 between the sensor controllers 300 and the main controller 400 than between the sensor elements 200 and the sensor controllers 300.

The positioning of the sensor controllers 300 in proximity to the sensor elements 200, at the distal end 110 in some embodiments described above, reduces the number of leads 750 or other connection elements that are required to travel the full length L of the catheter 100 to the main controller 400, allowing more sensor elements 200 to be used to measure more physical quantities without exceeding the strict dimensional requirements of the catheter 100. Converting the sensor signal 220 into the digital signal 332 in the sensor controller 300, and transmitting the digital signal 332 along the length L of the catheter 100 to the main controller 400, also reduces noise and maintains a high signal-to-noise ratio, resulting in higher quality sensor data reaching the main controller 400.

1. A catheter sensor array, comprising:
a catheter;
a plurality of sensor elements positioned at a distal end of the catheter; and
a plurality of sensor controllers each connected to at least one of the sensor elements by a first connection, each of the sensor controllers is positioned along a length of the catheter closer to the distal end of the catheter than to a proximal end of the catheter opposite the distal end.

2. The catheter sensor array of claim 1, further comprising a main controller connected to each of the sensor controllers by a second connection.

3. The catheter sensor array of claim 2, wherein a number of the second connections in the catheter sensor array is less than a number of the first connections in the catheter sensor array.

4. The catheter sensor array of claim 2, wherein the main controller is positioned at the proximal end of the catheter.

5. The catheter sensor array of claim 2, wherein each of the sensor controllers receives a sensor signal from the at least one of the sensor elements to which the sensor controller is connected and outputs a digital signal to the main controller.

6. The catheter sensor array of claim 1, wherein each of the sensor controllers is positioned at the distal end of the catheter.

7. The catheter sensor array of claim 1, wherein each of the sensor controllers is positioned within a proximity of the at least one of the sensor elements to which the sensor controller is connected, the proximity is less than a largest dimension of the sensor controller.

8. The catheter sensor array of claim 1, wherein at least one of the sensor controllers is connected to a subset of the plurality of sensor elements, the subset includes more than one of the plurality of sensor elements.

9. The catheter sensor array of claim 8, wherein the sensor elements of the subset measure different physical quantities.

10. The catheter sensor array of claim 1, wherein each of the sensor elements measures a pressure, a force, or a temperature.

11. The catheter sensor array of claim 2, wherein the sensor controllers are connected to the main controller in a daisy chain.

12. The catheter sensor array of claim 5, wherein the sensor controllers are connected to the main controller by a digital bus.

13. The catheter sensor array of claim 1, wherein the sensor elements and the sensor controllers are disposed on a flexible substrate, the first connection is a plurality of traces on the flexible substrate.

14. The catheter sensor array of claim 13, wherein the flexible substrate extends to the proximal end of the catheter.

15. The catheter sensor array of claim 13, wherein at least one of the sensor elements is connected to the flexible substrate by a solder bump on a surface of the flexible substrate.

16. The catheter sensor array of claim 15, wherein the at least one of the sensor elements has a through silicon via connected to the solder bump.

17. The catheter sensor array of claim 2, wherein the second connection is a wired connection or a wireless connection.

18. The catheter sensor array of claim 2, wherein the catheter has a handle at the proximal end, a plurality of arms at the distal end, and a lumen extending between the handle and the plurality of arms.

19. The catheter sensor array of claim 18, wherein the sensor elements, the sensor controllers, and the first connections are positioned in the arms.

20. The catheter sensor array of claim 18, wherein the second connection extends through the lumen to the main controller positioned in the handle.
